(19) United States
(12) Patent Application Publication
(10) Pub. No.: US 2018/0319786 A1
(43) Pub. Date: Nov. 8, 2018

(54) INHIBITOR OF THE P2X7 RECEPTOR

(71) Applicant: H. Lundbeck A/S, Valby (DK)

(72) Inventors: John Paul KILBURN, Haslev (DK); Allen T. HOPPER, Katonah, NY (US); Martin JUHL, Virum (DK)

(21) Appl. No.: 15/772,702

(22) PCT Filed: Nov. 1, 2016
(86) PCT No.: PCT/EP2016/076285

§ 371 Date: May 1, 2018

(30) Foreign Application Priority Data
Nov. 2, 2015 (DK) PA 2015 00678

(51) Int. Cl. C07D 417/14; A61P 29/00

(52) U.S. Cl. CPC C07D 417/14; A61P 29/00

(57) ABSTRACT

The present invention provides (S)-N-(2-(4-chlorophenyl)-2-morpholinoethyl)-2-(pyrimidin-2-yl)-4-(trifluoro-methyl)thiazole-5-carboxamide as P2X7 inhibitor, its use as a medicament, and a convenient enantiomeric synthesis protocol for the preparation of (S)-N-(2-(4-chlorophenyl)-2-morpholinoethyl)-2-(pyrimidin-2-yl)-4-(trifluoromethyl)thiazole-5-carboxamide.

### FIELD OF THE INVENTION

The present invention is directed to a novel compound which inhibits the P2X₇ receptor. Separate aspects of the invention are directed to pharmaceutical compositions comprising said compound and uses of the compound to treat pain, inflammation, neurological disorders, or neuropsychiatric disorders. In a further aspect the invention provides a convenient enantiomeric synthesis protocol for the preparation of (S)-N-(2-(4-chlorophenyl)-2-morpholinoethyl)-2-(pyrimidin-2-yl)-4-(trifluoromethyl)thiazole-5-carboxamide.

### BACKGROUND ART

The purinergic 2X7 (P2X₇) receptor is a ligand-gated ion channel which is activated by extracellular ATP and is present on a variety of cell types, including microglia in the central nervous system and other cells involved in inflammation and immune system function. The P2X₇ receptor has been shown to have a role in cytolysis in the immune system (Surprenant, et al. Science, 272, 735-41, 1996), and is involved in activation of lymphocytes and monocyte/macrophages leading to the increased release of pro-inflammatory cytokines (e.g., TNFα and IL1β) from these cells (Ferrari, et al. Neuropharmacol, 36, 1295-301, 1997). Studies have shown that inhibiting P2X₇ receptor activation in situations of inflammation (e.g., rheumatoid arthritis and other autoimmune diseases, osteoarthritis, asthma, chronic obstructive pulmonary disease and inflammatory bowel disease) or interstitial fibrosis results in a therapeutic effect (DiVirgilio, et al. Drug Dev Res, 45, 207-13, 1998). These and other studies indicate that P2X₇ receptor antagonists may find use in the treatment and prophylaxis of pain, including acute, chronic and neuropathic pain (Chessel, et al, Pain, 114, 386-96, 2005).

Inhibiting P2X₇ activation may also diminish or reduce cell death caused by prolongation of activated P2X₇ receptors, indicating a potential therapeutic intervention for said antagonists in nervous system injury or degeneration (Sperlagh, et al., Progress in Neurobiology, 7, 327-346, 2006). Vianna, et al. (Epilepsia, 43, 27-229, 2002) also revealed a potential role for P2X₇ receptors in the pathogenesis of epilepsy. Interestingly, because of the P2X₇ receptor's role in microglia activation and proliferation in the central nervous system (CNS), a self-propagating cycle of neuroinflammation and neurodegeneration results from P2X₇ receptor activation in areas of the brain (Monif, et al, J Neurosci, 29, 3781-91, 2009).

Thus, P2X₇ receptor antagonists, particularly small molecules with sufficient brain-penetrable properties, are desirable as useful agents for therapeutic intervention in the central nervous system for treating pain, inflammation, neurological and neurodegenerative disorders, neuropsychiatric disorders, or other disorders for which the reduction or otherwise stabilization of pro-inflammatory cytokines is beneficial. (±)-N-(2-(4-chlorophenyl)-2-morpholinoethyl)-2-(pyrimidin-2-yl)-4-(trifluoromethyl)thiazole-5-carboxamide in the racemic form has previously been described in Li et. al. WO 2009/012482, but the present invention provides a more potent enantiomeric form of the previously disclosed racemic mixture.

### SUMMARY OF THE INVENTION

An objective of the present invention is to provide a compound that inhibits the P2X₇ receptor. Accordingly, the present invention relates to the following (S)-N-(2-(4-chlorophenyl)-2-morpholinoethyl)-2-(pyrimidin-2-yl)-4-(trifluoromethyl)thiazole-5-carboxamide (I).

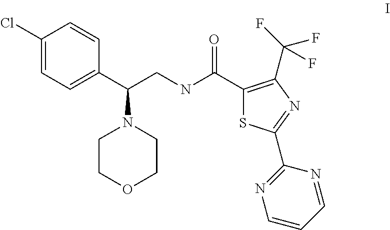 [SMILES: O=C(NC[C@H](c1ccc(Cl)cc1)N1CCOCC1)c1sc(-c2ncccn2)nc1C(F)(F)F]

The invention also provides a pharmaceutical composition comprising (S)-N-(2-(4-chlorophenyl)-2-morpholinoethyl)-2-(pyrimidin-2-yl)-4-(trifluoromethyl)thiazole-5-carboxamide or a pharmaceutically acceptable salt thereof, and optionally a pharmaceutically acceptable carrier, excipient or diluent.

The present invention further provides methods for treating pain or inflammation in a subject, comprising administering to a subject suffering from pain or inflammation a therapeutically effective amount of (S)-N-(2-(4-chlorophenyl)-2-morpholinoethyl)-2-(pyrimidin-2-yl)-4-(trifluoromethyflthiazole-5-carboxamide.

The present invention further provides methods for treating an affective disorder in a subject comprising administering to a subject suffering from an affective disorder a therapeutically effective amount of (S)-N-(2-(4-chlorophenyl)-2-morpholinoethyl)-2-(pyrimidin-2-yl)-4-(trifluoromethyflthiazole-5-carboxamide.

The present invention further provides methods for treating a neurological disorder or neurodegenerative disorder in a subject comprising administering to a subject suffering from a neurological disorder or neurodegenerative disorder a therapeutically effective amount of (S)-N-(2-(4-chlorophenyl)-2-morpholinoethyl)-2-(pyrimidin-2-yl)-4-(trifluoromethyl)thiazole-5-carboxamide.

The present invention further provides methods for treating depression, major depressive disorder, treatment resistant depression, anxiety, obsessive-compulsive disorder, post-traumatic stress disorder (PTSD), neuropathic pain, osteoarthritis, rheumatoid arthritis, psoriatic arthritis, inflammatory bowel disease, multiple sclerosis, epilepsy, Parkinson's Disease, Huntington's Disease and Alzheimer's disease, which involves administering (S)-N-(2-(4-chlorophenyl)-2-morpholinoethyl)-2-(pyrimidin-2-yl)-4-(trifluoromethyl)thiazole-5-carboxamide.

The present invention also provides the use of (S)-N-(2-(4-chlorophenyl)-2-morpholinoethyl)-2-(pyrimidin-2-yl)-4-(trifluoromethyl)thiazole-5-carboxamide for the manufacture of a medicament for the treatment of affective disorders selected from group consisting of depression, major depressive disorder, treatment resistant depression, anxiety, obsessive-compulsive disorder, post-traumatic stress disorder (PTSD), neuropathic pain, osteoarthritis, rheumatoid arthritis, psoriatic arthritis, inflammatory bowel disease, multiple sclerosis, epilepsy, Parkinson's Disease, Huntington's Disease and Alzheimer's disease.

The present invention also provides (S)-N-(2-(4-chlorophenyl)-2-morpholinoethyl)-2-(pyrimidin-2-yl)-4-(trifluoromethyl)thiazole-5-carboxamide for use in treating a disorder selected from depression, major depressive disorder, treatment resistant depression, anxiety, obsessive-compulsive disorder, post-traumatic stress disorder (PTSD), neuropathic pain, osteoarthritis, rheumatoid arthritis, psoriatic arthritis, inflammatory bowel disease, multiple sclerosis, epilepsy, Parkinson's Disease, Huntington's Disease and Alzheimer's disease in a subject.

These and other aspects of the invention will become apparent upon reference to the following detailed description.

### DETAILED DESCRIPTION OF THE INVENTION

The present invention is based on the discovery of (S)-N-(2-(4-chlorophenyl)-2-morpholinoethyl)-2-(pyrimidin-2-yl)-4-(trifluoromethyl)thiazole-5-carboxamide as a much more potent inhibitor of the P2X₇ receptor compared to (R)-N-(2-(4-chlorophenyl)-2-morpholinoethyl)-2-(pyrimidin-2-yl)-4-(trifluoromethyl)thiazole-5-carboxamide as well as the racemic mixture (±)-N-(2-(4-chlorophenyl)-2-morpholinoethyl)-2-(pyrimidin-2-yl)-4-(trifluoromethyl)thiazole-5-carboxamide. As such the compound of the present invention is particularly useful for the treatment of P2X₇ receptor related disorders. Additionally, certain aspects of the invention are explained in greater detail below but this description is not intended to be a detailed catalogue of all the different ways in which the invention may be implemented, or all the features that may be added to the instant invention. Hence, the following specification is intended to illustrate some embodiments of the invention, and not to exhaustively specify all permutations, combinations and variations thereof.

As used herein, the phrase “effective amount” when applied to a compound of the invention, is intended to denote an amount sufficient to cause an intended biological effect. The phrase “therapeutically effective amount” when applied to a compound of the invention is intended to denote an amount of the compound that is sufficient to ameliorate, palliate, stabilize, reverse, slow or delay the progression of a disorder or disease state, or of a symptom of the disorder or disease. In an embodiment, the method of the present invention provides for administration of combinations of compounds. In such instances, the “effective amount” is the amount of the combination sufficient to cause the intended biological effect.

The term “treatment” or “treating” as used herein means ameliorating or reversing the progress or severity of a disease or disorder, or ameliorating or reversing one or more symptoms or side effects of such disease or disorder. “Treatment” or “treating”, as used herein, also means to inhibit or block, as in retard, arrest, restrain, impede or obstruct, the progress of a system, condition or state of a disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of a disease or disorder state, amelioration or palliation of a disease or disorder state, and remission of a disease or disorder, whether partial or total, detectable or undetectable.

Pharmaceutically Acceptable Salts

The present invention also comprises salts of the present compounds, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines (for example, 8-bromotheophylline and the like). Further examples of pharmaceutically acceptable inorganic or organic acid addition salts include the pharmaceutically acceptable salts listed in S. M. Berge, et al., J. Pharm. Sci., 1977, 66, 2.

Furthermore, the compound of this invention may exist in unsolvated as well as in solvated forms with pharmaceutically acceptable solvents such as water, ethanol and the like.

Pharmaceutical Compositions

The present invention further provides a pharmaceutical composition comprising a therapeutically effective amount of (S)-N-(2-(4-chlorophenyl)-2-morpholinoethyl)-2-(pyrimidin-2-yl)-4-(trifluoromethyl)thiazole-5-carboxamide and a pharmaceutically acceptable carrier. The present invention also provides a pharmaceutical composition comprising a therapeutically effective amount of one of the specific compound disclosed in the Experimental Section and a pharmaceutically acceptable carrier.

The compound of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 22nd Edition, Gennaro, Ed., Mack Publishing Co., Easton, Pa., 2013.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general condition and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

The term “inhibit” or “inhibiting” as used herein means to reduce, diminish, block or even eliminate, such as in e.g. “inhibiting P2X₇ receptor activity”. “Inhibiting P2X₇ receptor activity” or “inhibiting P2X₇ activity” as used herein means, e.g. reducing or even eliminating the ability of a P2X₇ receptor to exhibit a cellular response, such as inhibiting the response to stimuli or agonist ligands, or inhibiting the production or accumulation of IL1β.

The present invention also provides a method of treating a disease or disorder, the method comprising administering a therapeutically effective amount of the compound of the present invention or a pharmaceutically acceptable salt thereof to a mammal suffering from (or at risk for) the disease or disorder, or otherwise in need of the treatment. The present invention also provides a method of treating pain or inflammation, the method comprising administering a therapeutically effective amount of the compound of the present invention or a pharmaceutically acceptable salt thereof to a mammal in need thereof. In an embodiment, the pain that may be treated using the compound described herein, including acute, chronic or inflammatory pain, is caused by neuropathic pain, post-operative pain, morphine tolerance, fibromyalgia, neuralgias, headache, osteoarthritis, rheumatoid arthritis, psoriatic arthritis, irritable bowel syndrome or inflammatory bowel disease.

In other embodiments, the disease or disorder that may be treated using the compound described herein is a neurological disorder or neurodegenerative disorder, such as epilepsy, multiple sclerosis, Parkinson's Disease, Huntington's Disease or Alzheimer's Disease. As used herein, the term “neurological disorder” means a disorder of the nervous system, and includes, but is not limited to, the disorders as described hereinabove. Based on the well-known meaning of disorders of the nervous system, neurological disorders result from structural, biochemical, electrical, or cellular (neuronal or microglial) signalling abnormalities that may occur in the brain or spinal cord of the afflicted mammal As used herein, the term “neurodegenerative disorder” means a disorder characterized by symmetrical and progressive loss of structure or function of neurons, such as death of neurons or reduced growth of neurons. Such loss of neurons may affect motor, sensory, or cognitive neuronal systems. As such, treating a neurological or neurodegenerative disorder using the compound described herein may result in the amelioration or relief of symptoms of the neurological or neurodegenerative disorder, such symptoms as paralysis, muscle weakness, poor coordination, uncontrolled movements, seizures, confusion, altered levels of consciousness, memory loss, emotional instability, loss of sensation, pain, and similar symptoms.

In an embodiment, the disease or disorder is a neuropsychiatric disorder, such as an affective disorder. As used herein, “affective disorder” means a mental disorder characterized by a consistent, pervasive alteration of mood, and affecting thoughts, emotions and behaviours. Affective disorders include mood disorders as described in DSM-IV-TR® (American Psychiatric Association, 2000, Diagnostic and Statistical Manual of Mental Disorders (4th ed., text rev.) doi:10.1176/appi.books.9780890423349; which is incorporated by reference herein). As such, treating an affective disorder using the compound described herein may result in the amelioration, stabilization or otherwise diminishment or relief of symptoms of the affective disorder, such symptoms as mood instability, manic episodes, feelings of guilt or worthlessness, sleep disturbances, agitation, or the like. Examples of affective disorders include, but are not limited to, depressive disorders, anxiety disorders, bipolar disorders, dysthymia and schizoaffective disorders. Anxiety disorders include, but are not limited to, generalized anxiety disorder, panic disorder, obsessive-compulsive disorder, phobias, and post-traumatic stress disorder (PTSD). Depressive disorders include, but are not limited to, major depressive disorder (MDD), catatonic depression, melancholic depression, atypical depression, psychotic depression, postpartum depression, treatment-resistant depression, bipolar depression, including bipolar I and bipolar II, and mild, moderate or severe depression. Personality disorders include, but are not limited to, paranoia, antisocial and borderline personality disorders.

In an embodiment of the invention, the affective disorder treated using the compound described herein is depression, major depressive disorder (MDD), treatment-resistant depression, bipolar disorder, generalized anxiety disorder, panic disorder, obsessive-compulsive disorder, or post-traumatic stress disorder (PTSD), or a combination thereof.

The present invention provides a method of treating an affective disorder in a subject, comprising administering to a subject in need of such treatment a therapeutically effective amount (S)-N-(2-(4-chlorophenyl)-2-morpholinoethyl)-2-(pyrimidin-2-yl)-4-(trifluoromethyl)thiazole-5-carboxamide.

The present invention provides a method of inhibiting P2X₇ activity in a subject, comprising administering to a subject in need thereof a therapeutically effective amount of (S)-N-(2-(4-chlorophenyl)-2-morpholinoethyl)-2-(pyrimidin-2-yl)-4-(trifluoromethyl)thiazole-5-carboxamide.

The present invention also provides a method of inhibiting production or accumulation of IL1β, comprising administering to a subject in need of such treatment a therapeutically effective amount of (S)-N-(2-(4-chlorophenyl)-2-morpholinoethyl)-2-(pyrimidin-2-yl)-4-(trifluoromethyl)thiazole-5-carboxamide.

In an embodiment, the present invention provides the use of a compound of Formula I for the manufacture of a medicament for the treatment of affective disorders. The present invention also provides the use of a compound of Formula I for the manufacture of a medicament for the inhibition of P2X₇ activity. The present invention further provides the use of (S)-N-(2-(4-chlorophenyl)-2-morpholinoethyl)-2-(pyrimidin-2-yl)-4-(trifluoromethyl)thiazole-5-carboxamide for the manufacture of a medicament for the inhibition of production or accumulation of IL1β.

In an embodiment, the present invention provides at least one compound of Formula I for use in treating an affective disorder in a subject. In an embodiment, the present invention provides (S)-N-(2-(4-chlorophenyl)-2-morpholinoethyl)-2-(pyrimidin-2-yl)-4-(trifluoromethyl)thiazole-5-carboxamide for use in inhibiting P2X₇ activity in a subject. In an embodiment, the present invention provides (S)-N-(2-(4-chlorophenyl)-2-morpholinoethyl)-2-(pyrimidin-2-yl)-4-(trifluoromethyl)thiazole-5-carboxamide for use in inhibiting production or accumulation of IL1β in a subject.

The invention also provides (S)-N-(2-(4-chlorophenyl)-2-morpholinoethyl)-2-(pyrimidin-2-yl)-4-(trifluoromethyl)thiazole-5-carboxamide for use in therapy of a subject, for example, in the treatment of affective disorders.

Experimental Section

(±)-N-(2-(4-chlorophenyl)-2-morpholinoethyl)-2-(pyrimidin-2-yl)-4-(trifluoromethyl)thiazole-5-carboxamide in the racemic form has previously been described in Li et. al. WO 2009/012482. Herein we describe a convenient enantiomeric synthesis protocol for the preparation of (S)-N-(2-(4-chlorophenyl)-2-morpholinoethyl)-2-(pyrimidin-2-yl)-4-(trifluoromethyl)thiazole-5-carboxamide.

General Methods

Analytical LC-MS and HPLC data were obtained using one of the methods identified below.

Method A: System controller shimadzu CBM-20A, UV detector shimadzu SPD-M20A. Column: C-18 4.6×30 mm 3.5=μm Symmetry, Column temperature: 60° C.

Mobile phase Gradient: Solvent A: H2O with 0.05% v/v TFA and Solvent B: Methanol with 0.05% TFA. Flow: 3.0 ml/minute.

Method B: A Waters Acquity UPLC-MS was used. Column: Acquity UPLC BEH C18 1.7 μm; 2.1×50 mm; Column temperature: 60° C.; Solvent system: A=water/trifluoroacetic acid (99.95:0.05) and B=acetonitrile/water/trifluoroacetic acid (94.965:5:0.035); Method: Linear gradient elution with A:B=90:10 to 0:100 in 1.0 minutes and with a flow rate of 1.2 mL/minute.

Method C: HPLC Column: Merck LiChroCart 250-4, LiChroSorb RP-8, 5μ, 250×4.6 mm Mobile phase: 20 mM triethylamine/phosphate buffer in water/acetonitrile 50/50, pH=3.0. Flow: 1.0 mL/minute. Temperature: 30° C.

Method D: Chiral HPLC Column: Chiralpak AD-H, 5 my, 250×4.6 mm

Eluent: Heptane:Ethanol:Diethylamine=90:10:0.1, flow 1.0 mL/minute, Temperature: 30° C.

¹H NMR spectra were recorded at 500 or 600 MHz on Bruker Avance instruments. TMS was used as internal reference standard. Chemical shift values are expressed in ppm. The following abbreviations are used for multiplicity of NMR signals: s=singlet, d=doublet, t=triplet, q=quartet, m=multiplet.

Abbreviations are in accordance with to the ACS Style Guide: “The ACS Style guide—A manual for authors and editors” Janet S. Dodd, Ed. 1997, ISBN: 0841234620.

### Preparation of (S)-N-(2-(4-chlorophenyl)-2-morpholinoethyl)-2-(pyrimidin-2-yl)-4-(trifluoromethyl)thiazole-5-carboxamide

In order to synthesize (S)-N-(2-(4-chlorophenyl)-2-morpholinoethyl)-2-(pyrimidin-2-yl)-4-(trifluoromethyl)thiazole-5-carboxamide in sufficient amounts, development of scalable routes towards the two coupling partners, thiazole carboxylic acid 5 and diamine 9 were developed. The synthesis of thiazole carboxylic acid 5 began with the formation of pyrimidine-2-carbothioamide 2, from the corresponding nitrile 1, using thioacetamide as the sulfur source in an aqueous acidic DMF solution affording pyrimidine-2-carbothioamide 2 in 82% yield. Subsequently, Hantzsch thiazole synthesis was used to prepare the heteroaromatic five-membered ringsystem in a modest 32% by subjecting pyrimidine-2-carbothioamide 2 to 2-Chloro-4,4,4-trifluoroacetoacetic acid ethyl ester 3 in DMF at 110° C. Subsequent exposure to lithium hydroxide in a THF:MeOH:water mixture produced the desired carboxylic acid coupling partner 5 via hydrolysis in 96% yield.

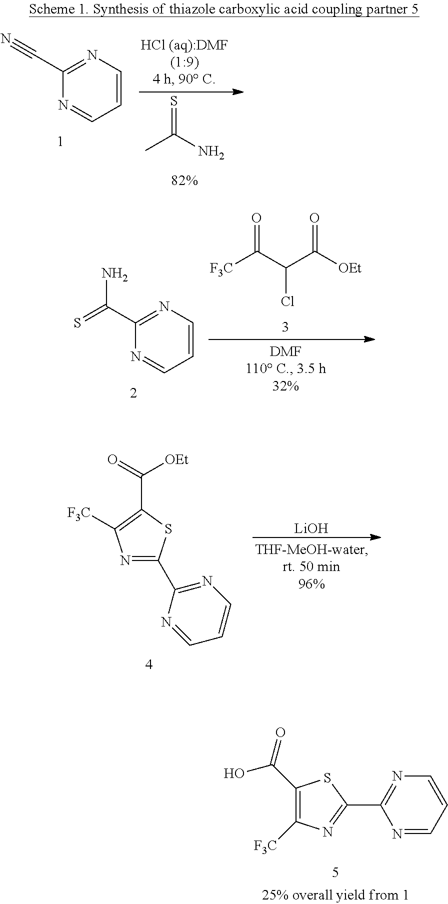

The optimized synthesis of the enantiomerically enriched diamine 9 initiated with a 1,4-conjugated addition of morpholine to nitroolefin 6, and the corresponding adduct precipitated directly by the addition of 2M hydrochloric acid. The resulting white solid, was isolated via filtration of the reaction mixture affording the hydrochloride salt 7. Attempts to dry hydrochloride 7 in a vacuum oven at 40° C., resulted in elimination of morpholine affording back nitroolefin 6, therefore the hydrochloride salt 7 was used directly. Reduction of the nitro-moiety was achieved by exposure to zinc in an aqueous ethanolic solution, thus providing racemic diamine 8 in 87% yield. After extensive screening an enantioselective resolution of diamine 8 using L-N-acetyl-leucine in a mixture of acetonitrile and water was discovered affording the S-enantiomer in 30% yield and >97% ee.

The diamine was subsequently coupled with the corresponding acid chloride of carboxylic acid 5, thereby affording (S)-N-(2-(4-chlorophenyl)-2-morpholinoethyl)-2-(pyrimidin-2-yl)-4-(trifluoromethyl)thiazole-5-carboxamide in 26% overall yield, 99% UV purity, and >97% ee.

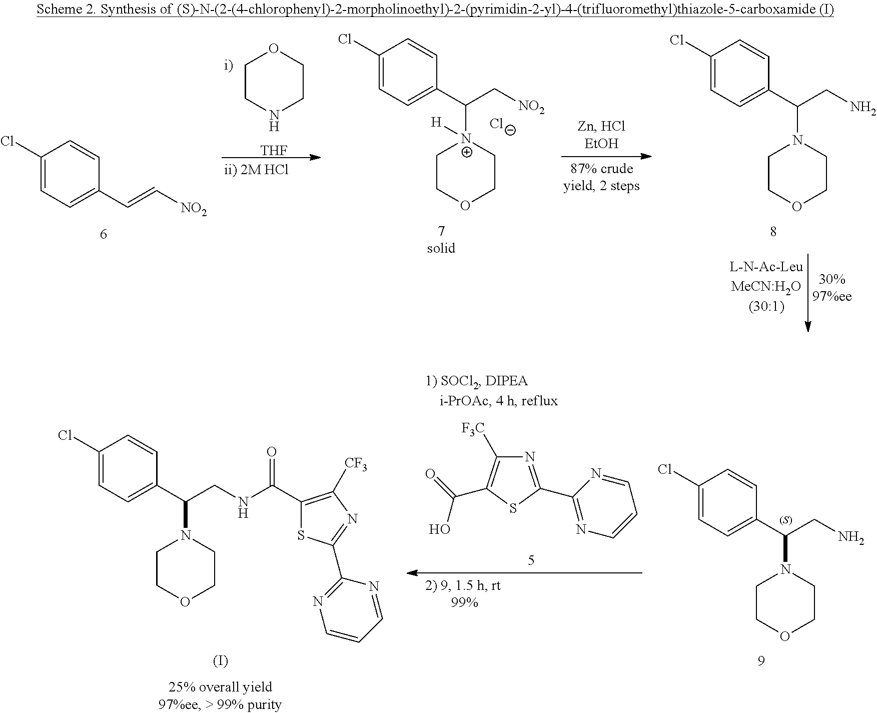

The synthesis of 2 S)-N-(2-(4-chlorophenyl)-2-morpholinoethyl)-2-(pyrimidin-2-yl)-4-(trifluoromethyl)thiazole-5-carboxamide is described in details in the Example 1-7 below.

Example 1 discloses a convenient synthesis protocol for the preparation of the coupling partner 2-(Pyrimidin-2-yl)-4-(trifluoromethyl)thiazole-5-carboxylic acid 5.

Examples 2-7 disclose a convenient enantiomeric synthesis protocol for the preparation of (S)-N-(2-(4-chlorophenyl)-2-morpholinoethyl)-2-(pyrimidin-2-yl)-4-(trifluoromethyl)thiazole-5-carboxamide.

### Example 1

### 2-(Pyrimidin-2-yl)-4-(trifluoromethyl)thiazole-5-carboxylic acid

Pyrimidine-2-carbothioamide 2 was produced in 82% yield from corresponding nitrile 1 using thioacetamide as a sulfur source and heating in an aqueous acidic DMF solution. Subsequently, the Hantzsch thiazole synthesis method was employed to give the heteroaromatic five-membered ring system 4 in 32% yield by reacting pyrimidine-2-carbothioamide 2 with 2-chloro-4,4,4-trifluoroacetoacetic acid ethyl ester 3 in DMF at 110° C. Ester hydrolysis of 4 using lithium hydroxide in a THF:MeOH:water mixture produced the desired thiazole carboxylic acid coupling partner 5 in 96% yield.

### Example 2

### 1-Chloro-4-((E)-2-nitro-vinyl)-benzene

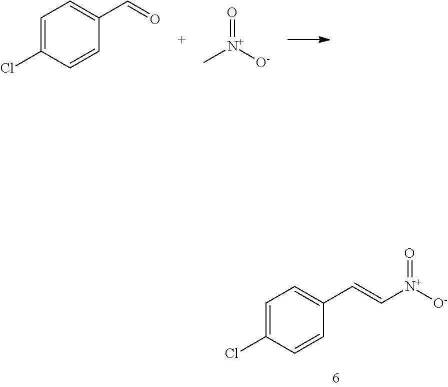

4-Chlorobenzaldehyde (200.0 g, 1423 mmol) was dissolved in acetic acid (600.0 mL, 10550 mmol) and nitromethane (308.2 mL, 5691 mmol) was added, then acetic anhydride (26.85 mL, 284.6 mmol) and ammonium acetate (120.6 g, 1565 mmol) was added. The reaction was heated at reflux for 2 hr. The volatiles were removed by evaporation, water was added (400 mL, 20000 mmol) followed by ethyl acetate (400 mL, 4000 mmol). The solids were filtered and dried to yield the title compound 6 as a brown solid (178.8 g). ¹H NMR (CDCl₃ 500 MHz): δppm 6.88 (d, 1H), 6.48 (d, 1H), 6.47-6.32 (m, 4H). HPLC, t_{R} (minutes, Method A)=1.48.

### Example 3

### 4-[1-(4-Chloro-phenyl)-2-nitro-ethyl]-morpholine, hydrochloride salt

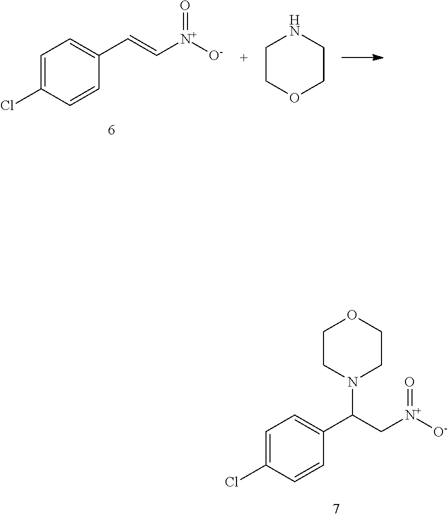 [SMILES: O=[N+]([O-])/C=C/c1ccc(Cl)cc1 ; C1COCCN1 ; O=[N+]([O-])CC(c1ccc(Cl)cc1)N1CCOCC1]

1-Chloro-4-((E)-2-nitro-vinyl)-benzene 6 (178.8 g, 973.9 mmol) was dissolved in tetrahydrofuran (383.9 mL, 4733 mmol). Morpholine (84.93 mL, 973.9 mmol) was added and the mixture stirred for 30 minutes at RT. Diethyl ether (1329 mL, 12660 mmol) was added followed by 2.0 M hydrogen chloride in diethyl ether (584.3 mL). Internal temperature rose to 36° C. and the slurry was stirred for 15 minutes before being cooled on an ice bath with stirring for 15 minutes. The solids were filtered and washed with ether. The resulting title compound 7 was used without further purification in the next step.

### Example 4

### 2-(4-Chloro-phenyl)-2-morpholin-4-yl-ethylamine

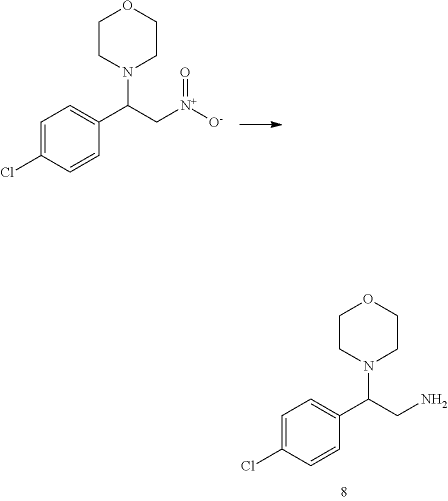 [SMILES: O=[N+]([O-])CC(c1ccc(Cl)cc1)N1CCOCC1 ; NCC(c1ccc(Cl)cc1)N1CCOCC1]

4-[1-(4-Chloro-phenyl)-2-nitro-ethyl]-morpholine, hydrochloride salt 7 (59.0 g, 192 mmol) was suspended in ethanol (680 mL, 12000 mmol), then 37% aqueous HCl solution (200 mL) was added followed by the addition of zinc (42.8 g, 654 mmol) in 4 portions. The mixture was stirred for 50 min during which the mixture became a clear almost colourless solution. The volatiles were removed by evaporation and the aqueous mixture was quenched by pouring into 32% aqueous ammonia solution (400 mL). The organics were extracted with iPrOAc (2×100 mL). The combined organic phases were dried (sodium sulfate), filtered, and evaporated to dryness in vacuo using MeCN (3×100 mL) to co-evaporate affording compound 8 (45.1 g). ¹H NMR (CDCl₃ 600 MHz): δppm 7.31 (d, 2H), 7.20 (d, 2H), 3.71-3.65 (m, 4H), 3.26 (t, 1H), 3.09-3.04 (m, 1H), 2.96-2.91 (m, 1H), 2.43-2.37 (m, 4H).

### Example 5

### (S)-2-(4-Chloro-phenyl)-2-morpholin-4-yl-ethylamine (S)-2-acetylamino-4-methyl-pentanoate

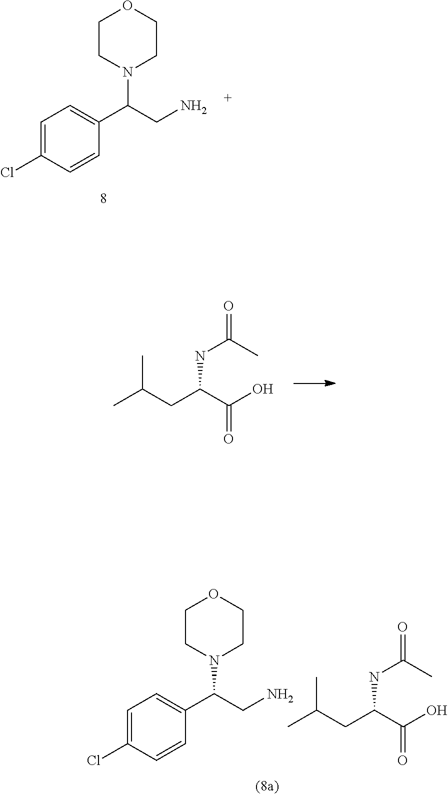

To a solution of 2-(4-chlorophenyl)-2-morpholinoethylamine 8 (106.70 g, 0.443 mol) dissolved in acetonitrile (3005 mL) was added (S)-2-acetamido-4-methylpentanoic acid (N-Acetyl-L-Leucine 76.80 g, 0.443 mol) and the mixture was heated to reflux and water (105 mL) was added giving a clear solution. The heating was stopped and the solution was allowed to cool slowly to room temperature. At ca. 45° C. crystallization started and was continued for 4 hours while cooling slowly to 20° C. The product was isolated by filtration and washed on the filter with acetonitrile (2×100 mL) and dried in vacuo. The solid was re-suspended in acetonitrile:water=96.5:3.5 (240 mL). The slurry was then heated slowly to reflux for 1 hour, heating was removed and stirring was continued while slowly cooling to room temperature. The slurry was stirred for 16 hours at RT. The precipitated compound was isolated by filtration, washed on the filter with acetonitrile:water=96.5/3.5 (2×20 mL) and acetonitrile (1×30 mL) and dried. The product was then dried to constant weight in vacuo at 40° C. to yield compound 8a as a white solid (55.1 g). ¹H NMR (CDCl₃ 600 MHz): δppm 7.68 (d, 1H), 7.44 (d, 2H), 7.26 (d, 2H), 4.08-4.03 (m, 1H), 3.71-3.68 (m, 1H), 3.58-3.52 (m, 4H), 3.38-3.32 (m, 1H), 2.88-2.83 (m, 1H), 2.40-2.35 (m, 2H), 2.22-2.15 (m, 2H), 1.81 (s, 3H), 1.62-1.55 (m, 1H), 1.50-1.45 (m, 1H), 1.41-1.36 (m, 1H), 0.85 (d, 3H), 0.81 (d, 3H). HPLC t_{R} (minutes, Method C)=2.53.

### Example 6

### (S)-2-(4-chlorophenyl)-2-morpholinoethylamine

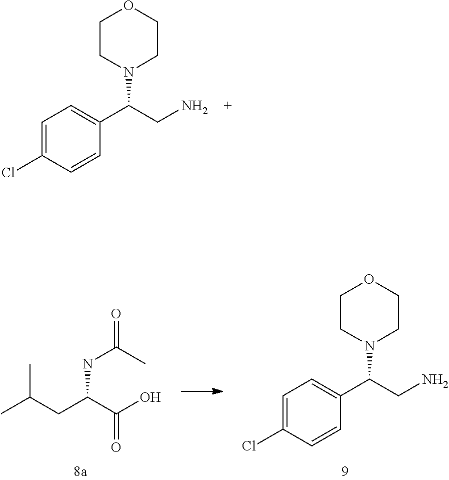

To a solution of 2-(4-chlorophenyl)-2-morpholinoethylamine (S)-2-acetamido-4-methylpentanoate 8a (55.1 g) in iPrOAc/H₂O (400 mL) was added 24% ammonia, aq. The phases were separated and the water phase was extracted once more with iPrOAc (50 mL) and the combined organic phases were washed with water (2×50 mL), dried over MgSO4 and the solvent was removed in vacuo. NaCl (ca. 20 g) was dissolved in the water phase, and then extracted with iPrOAc (3×100 mL). The combined organic phases were washed with brine (2×50 mL), dried over MgSO₄, evaporated and dried to constant weight to yield compound 9 (31.3 g). HPLC t_{R} (min, Method C)=2.53, Chiral HPLC t_{R} (minutes, Method D)=14.96.

### Example 7

### (S)-N-(2-(4-chlorophenyl)-2-morpholinoethyl)-2-(pyrimidin-2-yl)-4-(trifluoromethyl)thiazole-5-carboxamide

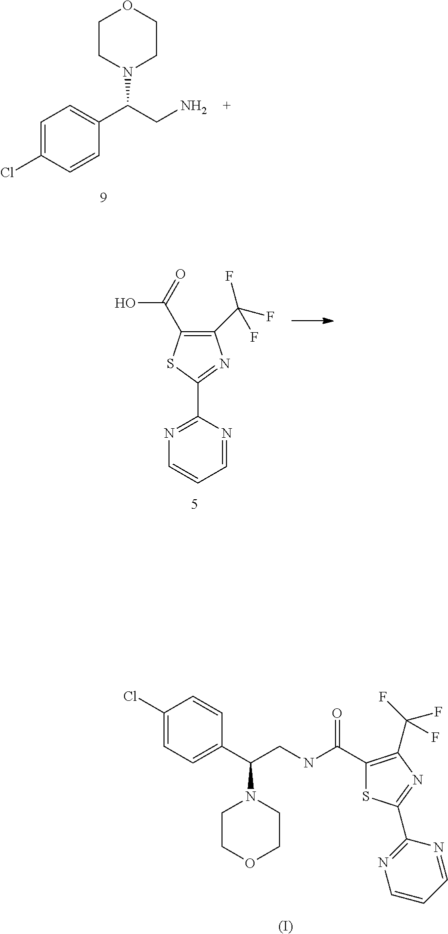

(S)-2-(4-chlorophenyl)-2-morpholinoethylamine 9 (31.3 g, 130 mmol) was dissolved in dry THF (700 mL) at RT. 2-(Pyrimidin-2-yl)-4-(trifluoromethyl)thiazole-5-carboxylic acid 5 (39.4 g, 143 mmol, preparation previously described in Li et. al. WO 2009/012482) was added followed by triethylamine (54.4 mL, 390 mmol). 2,4,6-Tripropyl-1,3,5,2,4,6-trioxatriphosphinane 2,4,6-trioxide (122 mL, 50% solution i ethyl acetate, dissolved in THF 100 mL) was added slowly and resulted quickly in a clear solution. The reaction was stirred at RT for 3 h. To the reaction was added iPrOAc (500 mL) and the organic phase was washed with 0.5 M NaOH (200 mL) and 0.1 M NaOH (200 mL) followed by washing with water (2×200 mL). The product was then extracted with 1M H₂SO₄ (4×200 mL) and the combined water-phases were washed with iPrOAc (2×200 mL) and was again made basic with 24% ammonia, aq. (strong exotherm, cooled with ice) and extracted with iPrOAc (2×400 mL). The combined organic phases were washed with water (1×300 mL) and brine (2×200 mL), dried over MgSO₄ and the solvent was partly removed in vacuo (volume ca. 400 mL) and heptane (600 mL) was added. The mixture was stirred on the rotary evaporator for 16 hours while slowly cooling to room temperature. The reaction was cooled on ice for 30 min and the product was isolated by filtration, washed with heptane (2×50 mL) and dried under vacuum to constant weight to yield the title compound (40 g). ¹H NMR (DMSO-d₆ 600 MHz): δppm 9.02 (d, 2H), 8.94 (t, 1H), 7.71 (t, 1H), 7.44 (d, 2H), 7.33 (d, 2H), 3.92-3.86 (m, 1H), 3.67-3.64 (m, 1H), 3.60-3.48 (m, 5H), 2.40-2.30 (m, 4H). LCMS (MH⁺): m/z=498.3, t_{R} (min, Method B)=0.49. HPLC t_{R} (min, Method C)=5.16. [α]²⁰,D=−34.89 (c=1% in MeOH). XRPD analysis continued that the obtained compound (I) is (S)-N-(2-(4-chlorophenyl)-2-morpholinoethyl)-2-(pyrimidin-2-yl)-4-(trifluoromethyl)thiazole-5-carboxamide.

P2X₇ FLIPR Assay

HEK293 cells stably expressing either human, mouse, or rat P2X7 receptors were plated in 384-well poly-D-lysine-coated black FLIPR plates (Greiner One, Cat #781946) using 1.5% FBS media 24 hours before the assay. Sucrose buffer, pH 7.4 was used for the human-P2X7 assay (5 mM of potassium chloride, 9.6 mM NaH₂PO₄.2H₂O, 25 mM Hepes, 0.5 mM CaCl₂, 5 mM glucose, 280 mM sucrose and 1.0 mM probenecid (Sigma, St. Louis, Mo.)). Hank's balanced salt solution buffer, pH 7.4 was used for the rat-P2X7 assay (1×HBSS buffer supplemented with 20 mM Hepes plus 2.5 mM probenecid (Sigma, St. Louis, Mo.) and 0.05% bovine serum albumin After removing the media, the plates were loaded with 30 μL of Fluo-4 NW (Molecular Probes, F36206), and were incubated at 37° C. for 30 min in a humidified chamber (5% CO2/95% air), and for 30 min at RT. Mobilization of intracellular Ca⁺² in response to different ligands was measured online using the FLIPR Tetra reader. In the assay, baseline fluorescence was measured for 15 seconds, then 15 μL of 4× compounds (40 μM) was added, fluorescence was monitored for 3 min for agonist activity, 15 μL of 4×BzATP (hP2X7 1×8 μM, rP2X7 1×15 μM) was added and after a 30 min incubation at room temperature the fluorescence was read for 3 min for IC₅₀ determination. The data was analyzed using Lundbeck LSP curve-fit software which is similar to Prism non-linear regression curve-fit analysis, results are shown in Table 1 and show that (S)-N-(2-(4-chlorophenyl)-2-morpholinoethyl)-2-(pyrimidin-2-yl)-4-(trifluoromethyl)thiazole-5-carboxamide is a more potent inhibitor of P2X₇ receptor than both the corresponding R-form ((R)-N-(2-(4-chlorophenyl)-2-morpholinoethyl)-2-(pyrimidin-2-yl)-4-(trifluoromethyl)thiazole-5-carboxamide) and the racemic mixture.

TABLE 1
P2X₇ receptor in vitro molecular pharmacology data for compound
(I) and comparator compounds: R-form and racemic mixture

| | P2X₇ FLIPR DATA |
|---|---|
| | IC₅₀ (NM) |
| Species | Human |
| Compound (I) | 12 ± 10 |
| Compound (II) | 89 ± 13 |
| Compound (III) | 32 ± 13 |

Compound (I): (S)-N-(2-(4-chlorophenyl)-2-morpholinoethyl)-2-(pyrimidin-2-yl)-4-(trifluoromethyl)thiazole-5-carboxamide
Compound (II): (R)-N-(2-(4-chlorophenyl)-2-morpholinoethyl)-2-(pyrimidin-2-yl)-4-(trifluoromethyl)thiazole-5-carboxamide
Compound (III): (±)-N-(2-(4-chlorophenyl)-2-morpholinoethyl)-2-(pyrimidin-2-yl)-4-(trifluoromethyl)thiazole-5-carboxamide

### EVALUATION OF (S)-N-(2-(4-CHLOROPHENYL)-2-MORPHOLINOETHYL)-2-(PYRIMIDIN-2-YL)-4-(TRIFLUOROMETHYL)THIAZOLE-5-CARBOXAMIDE EFFECT IN PAIN MODELS

(S)-N-(2-(4-chlorophenyl)-2-morpholinoethyl)-2-(pyrimidin-2-yl)-4-(trifluoromethyl)thiazole-5-carboxamide demonstrated efficacy in three models of pain in rats, including the complete Freund's adjuvant (CFA) model of inflammatory pain, the spared nerve injury (SNI) and complete constriction injury (CCI) models of neuropathic pain.

CFA Model of Inflammatory Pain

Animals for CFA and SNI Pain Experiments

Male Sprague Dawley rats (Charles River, Germany) weighing 250-300 g on the day of gabapentin administration were used (n=8-10 per group). The animals were housed in Macrolon III cages (20×14×18 cm or 20×40×18 cm; in groups of 3-5 per cage according to weight) containing wood-chip bedding material (3×1×4 mm) in an air-conditioned building with controlled environmental parameters (relative humidity 55±15%, temperature 20±2° C., and light from 06.00 to 19.00 h). Food and water were available ad libitum. The rats were allowed to habituate to the housing facilities for at least one week prior to being assigned to behavioral experiments whereupon they were randomly distributed across treatment groups. All experiments were performed according to the Ethical Guidelines of the International Association for the Study of Pain and the Danish Committee for Experiments on Animals.

CFA-Induced Inflammatory Hyperalgesia

Individual rats received a subcutaneous injection of CFA (50% in saline, 100 μl total volume, Sigma Aldrich) into the plantar surface of the hindpaw. All rats were then immediately returned to their homecage. The day prior to this (pre-CFA baseline response), and then again 24 h post CFA injection (post-CFA baseline response), hindpaw paw pressure thresholds were measured to obtain an index of evoked mechanical hyperalgesia [Ref: Munro, G., et al., Neuropharmacology, 2011. 61(1-2): p. 121-132.1. To do this, the investigator gently restrained the rat prior to the application of progressively increasing mechanical pressure to the mid hindpaw region using an electronic version of the Randall-Selitto device (HTC, U.S.A.) The point at which the rat attempted to make a reflex hindpaw withdrawal which in some instances was followed by vocalization was recorded as the paw pressure threshold (g). After 2-3 min a second measurement was taken from an adjacent region of the hindpaw. Thereafter, CFA inflamed rats received oral administration of (S)-N-(2-(4-chlorophenyl)-2-morpholinoethyl)-2-(pyrimidin-2-yl)-4-(trifluoromethyl)thiazole-5-carboxamide or vehicle with the investigator blinded to treatment and the paw pressure threshold measured again 60 minutes later.

Results: CFA-Induced Inflammatory Hyperalgesia

Hindpaw injection of CFA produced a robust inflammatory hyperalgesia as indicated by the reduction in paw pressure threshold to mechanical stimulation 24 h later (113±3 g vs 300±5 g pre-CFA, P<0.001, Students t-test). This mechanical hyperalgesia was significantly reversed by (S)-N-(2-(4-chlorophenyl)-2-morpholinoethyl)-2-(pyrimidin-2-yl)-4-(trifluoromethyl)thiazole-5-carboxamide treatment (F(4.49)=30.692, P<0.001). Notably, the efficacy obtained with the 30 mg/kg dose of (S)-N-(2-(4-chlorophenyl)-2-morpholinoethyl)-2-(pyrimidin-2-yl)-4-(trifluoromethyl)thiazole-5-carboxamide was approached that obtained with the NSAID diclofenac (62 vs 84% reversal respectively)

SNI Model of Neuropathic Pain

A spared nerve injury (SNI) was performed in rats (body weight 180-220 g at the time of surgery) as described previously [Decosterd, I. and C. J. Woolf, Pain, 2000. 87(2): p. 149-581. Anaesthesia was induced and maintained by 2% isoflurane (Baxter A/S, Alleroed, Denmark), combined with oxygen (30%) and nitrous oxide (68%). Next, the skin of the lateral left thigh was incised and the cranial and caudal parts of the biceps femoris muscle were separated and held apart by a retractor to expose the sciatic nerve and its three terminal branches: the sural, common peroneal and tibial nerves. The tibial and common peroneal nerves were tightly ligated with 4/0 silk and 2-3 mm of the nerve distal to the ligation was removed. Any stretching or contact with the intact sural nerve was avoided. The overlying muscle was closed in layers with 4/0 synthetic absorbable surgical suture and the skin closed and sutured with 4/0 silk thread. After 4-5 days rats were routinely tested for the presence of neuropathic hypersensitivity. This entailed removing them from their home cage and allowing them to habituate for 15 min in an openly ventilated 15×20 cm white Plexiglass testing cage which was placed upon an elevated metal grid allowing access to the plantar surface of the injured hindpaw. The presence of mechanical allodynia was assessed using a series of calibrated von Frey hairs (lower limit=0.06 and upper limit=13.5 g, Stoelting Co, Wood Dale Ill.), which were applied to the lateral aspect of the plantar surface of the hindpaw with increasing force until an individual filament used just started to bend. The filament was applied for a period of 1-2 s and was repeated 5 times at 1-2 s intervals. The filament that induced a reflex paw withdrawal in 3 out of 5 applications was considered to represent the threshold level for a mechanical allodynic response to occur. Only those animals showing distinct neuropathic behaviours from between 10-30 days post-surgery were used for assessing efficacy of (S)-N-(2-(4-chlorophenyl)-2-morpholinoethyl)-2-(pyrimidin-2-yl)-4-(trifluoromethyl)thiazole-5-carboxamide.

Results: SNI Model of Neuropathic Pain

Results showed that in SNI rats at 3-4 weeks post injury von Frey stimulation of the injured hindpaw revealed a pronounced mechanical hypersensitivity as compared with the PWT prior to injury (3.3±0.3 g vs 8.4±0.5 g, P<0.001, Students t-test). Administration of (S)-N-(2-(4-chlorophenyl)-2-morpholinoethyl)-2-(pyrimidin-2-yl)-4-(trifluoromethyl)thiazole-5-carboxamide (30 and 100 mg/kg) dose dependently reversed the mechanical hypersensitivity (F(3.47)=12.522, P<0.001) with efficacy comparable to that obtained with a 100 mg/kg dose of gabapentin (108 vs 132% reversal respectively and the PWT was almost fully restored to the pre-SNI level.

Chronic Constrictive Injury (CCI) Pain Model of Neuropathic Pain

Animals for Chronic Constrictive Injury (CCI) Pain Experiments

Male Sprague Dawley rats (Harlan) were used in the study. Upon receipt, rats were group housed with 2-3 per cage in standard cages. Rats were allowed to acclimate for up to 1 week prior to surgery. All rats were examined and weighed prior to initiation of the study to assure adequate health and suitability. During the course of the study, 12/12 light/dark cycles were maintained. The room temperature was maintained between 20° C. and 23° C. with a relative humidity maintained around 50%. Chow and water were provided ad libitum for the duration of the study. All testing was performed during the animals' light cycle phase. Rats were single housed after surgery.

Methods for Chronic Constrictive Injury (CCI) Model of Neuropathic Pain

This surgery was performed according to Bennett and Xie (1988). Specifically, rats were anesthetized with isoflurane (2% in Oxygen). The left hind flank was shaved and sterilized and the rat positioned on its side on a sterile surgical field. The pelvic bone ridge was palpated and a vertical incision was made perpendicular to the long axis of the spine. The first layer of muscle was cut to expose the sciatic nerve. Retractors were used to widen the incision, centering the portion of the sciatic nerve to be ligated. The exposed nerve was carefully teased apart from the second layer of muscle, removing fascia lining. Using 5 cm length of 4.0 chromic gut suture (pre-soaked in saline), 4 loose ligations were made around the sciatic nerve, spaced at 0.5 cm intervals. Sutures were positioned superior to the point where the nerve branches. The incision was then closed in layers, using 4.0 absorbable suture, and the skin closed using sterile autoclips. Topical antibiotic ointment was applied to the closed incision. Each rat was monitored until awake and moving freely around the recovery chamber. Animals were single-housed for the entire duration of the study.

Evaluation of Mechanical Allondynia

Paw withdrawal from a mechanical stimulus was measured by applying von Frey filaments of ascending bending force to the plantar surface of the hind paws (ipsilateral and contralateral). Baseline and post-treatment withdrawal threshold values for non-noxious mechanical sensitivity were evaluated using von Frey filaments (Semmes-Weinstein filaments, Stoelting, Wood Dale, Ill., USA) of varying stiffness (0.4, 0.7, 1.2, 2.0, 3.6, 5.5, 8.5, 10, 15, 26 g). Animals were placed on a perforated metallic platform and allowed to acclimate to their surroundings for a minimum of 15 minutes before each test session. Each filament was presented perpendicular to the plantar surface with sufficient force to cause slight buckling against the paw, then held until a positive response was noted (paw sharply withdrawn). Confirmation of threshold was tested by examining the filament above and below the threshold withdrawal response. Each filament was applied 3 times.

Prior to CCI surgery, rats were tested for baseline threshold using the von Frey filaments. Rats with a paw withdrawal threshold (PWT) less than 12 g were not included in the study. Rats were balanced based on their post-surgery PWT at approximately 2 weeks after surgery. Animals with a post-surgical PWT above 5.5 g were removed from in the study. On test day, rats were administered vehicle, gabapentin or test compound and PWT was assessed 120 minutes following drug administration.

Results: Chronic Constrictive Injury (CCI) Pain Model of Neuropathic Pain

Data were analyzed by Analysis of variance (ANOVA), followed by post-hoc comparisons when appropriate. An effect was considered significant at p<0.05 level.

The analysis showed that gabapentin (100 mg/kg) significantly increased PWT in CCI animals compared to vehicle treatment. (S)-N-(2-(4-chlorophenyl)-2-morpholinoethyl)-2-(pyrimidin-2-yl)-4-(trifluoromethyl)thiazole-5-carboxamide (100 mg/kg) significantly increased PWT compared to CCI animals treated with vehicle.

1. The compound (S)-N-(2-(4-chlorophenyl)-2-morpholinoethyl)-2-(pyrimidin-2-yl)-4-(trifluoromethyl)thiazole-5-carboxamide or a pharmaceutically acceptable salt thereof.

2. The compound of claim 1, wherein the compound is (S)-N-(2-(4-chlorophenyl)-2-morpholinoethyl)-2-(pyrimidin-2-yl)-4-(trifluoromethyl)thiazole-5-carboxamide.

3-5. (canceled)

6. A pharmaceutical composition comprising the compound of claim 1, and one or more pharmaceutically acceptable carriers, diluents and excipients.

7. A method of producing (S)-N-(2-(4-chlorophenyl)-2-morpholinoethyl)-2-(pyrimidin-2-yl)-4-(trifluoromethyl)thiazole-5-carboxamide comprising the steps:
a. Conversion of 2-(4-chlorophenyl)-2-morpholinoethylamine to (S)-2-(4-chlorophenyl)-2-morpholinoethylamine in the presence of (S)-2-acetamido-4-methylpentanoic acid, and
b. Reacting the obtained (S)-2-(4-chlorophenyl)-2-morpholinoethylamine with 2-(Pyrimidin-2-yl)-4-(trifluoromethyl)thiazole-5-carboxylic acid to obtain (S)-N-(2-(4-chlorophenyl)-2-morpholinoethyl)-2-(pyrimidin-2-yl)-4-(trifluoromethyl)thiazole-5-carboxamide.

8. A method of treating a patient suffering from acute, chronic or inflammatory pain, comprising administering to the patient the compound (S)-N-(2-(4-chlorophenyl)-2-morpholinoethyl)-2-(pyrimidin-2-yl)-4-(trifluoromethyl)thiazole-5-carboxamide, or a pharmaceutically acceptable salt thereof.

9. The method of claim 8, wherein the compound is (S)-N-(2-(4-chlorophenyl)-2-morpholinoethyl)-2-(pyrimidin-2-yl)-4-(trifluoromethyl)thiazole-5-carboxamide.

10. The method of claim 8, wherein the pain is caused by neuropathic pain, post-operative pain, morphine tolerance, fibromyalgia, neuralgias, headache, osteoarthritis, rheumatoid arthritis, psoriatic arthritis, irritable bowel syndrome or inflammatory bowel disease.
